(19) United States
(12) Patent Application Publication
(10) Pub. No.: US 2003/0109865 A1
(43) Pub. Date: Jun. 12, 2003

(54) Utilization of a multi-character material in a surface coating of an electrosurgical instrument

(75) Inventors: Darcy Wayne Greep, South Jordan, UT (US); William George Pitt, Orem, UT (US)

Correspondence Address: WORKMAN NYDEGGER & SEELEY, 1000 EAGLE GATE TOWER, 60 EAST SOUTH TEMPLE, SALT LAKE CITY, UT, 84111, US

(73) Assignee: MegaDyne Medical Products, Inc.

(21) Appl. No.: 10/021,607

(22) Filed: Dec. 12, 2001

(51) Int. Cl. A61B 18/18

(52) U.S. Cl. 606/41; 606/45

(57) ABSTRACT

Providing a multi-character material as part of a coating of an electrosurgical instrument. The multi-character material includes molecular chains having a hydrophobic characteristic or charged unit that bonds to a metallic substrate and/or a base material coating layer of the tip, and a hydrophilic characteristic that provides desired properties to the coated surface, such as the ability to attract water to assist in cooling and/or lubricating an electrosurgical tip, to create a low shear, sacrificial layer that protects and enhances eschar release characteristics, to supply a radical scavenger or inhibitor that reduces damage at the tip, and/or to deposit factors, such as healing factors, from the tip onto one or more contact surfaces of the patient's body. Optionally, an additional covering layer of a hydrophilic material may be applied over the multi-character material coating layer to increase the desired properties at the coated surface.

### RELATED PATENT APPLICATIONS

Reference is made to co pending U.S. patent application Ser. No. ______, entitled “Application and Utilization of a Water-Soluble Polymer on a Surface,” the disclosure of which is incorporated by this reference.

### BACKGROUND OF THE INVENTION

1. The Field of the Invention

The present invention relates to coated electrosurgical instruments. More particular, the present invention relates to using a multi-character material to coat at least a portion of a surface of an electrosurgical instrument in order to provide desirable properties, characteristics and/or attributes at the flesh-contacting surface.

2. The Relevant Technology

The application of a polymer as a coating layer on a surface has proved to be a valuable asset in a variety of applications. One such application includes the use of a fluoropolymer, such as polytetrafluoroethylene (“PTFE”), as a coating layer of a surface. PTFE is a durable, chemically resistant, nonflammable thermoplastic substance that is widely used to coat a variety of surfaces including metal surfaces.

In the area of manufacturing pans for cooking, a fluoropolymer, such as a Teflon®, can be used as a non-stick coating layer. The non-stick layer facilitates the removal of food and other debris from the pan surface.

In the area of electrosurgery, fluoropolymers have been used to surface coat at least a portion of an electrosurgical tip to provide additional properties to the tip, including providing non-stick surface and high temperature stability. Electrosurgery includes surgical procedures that use radio frequency (RF) electrical energy to cut tissue and/or cauterize blood vessels and/or tissues. The RF energy is produced by a signal generator and is transmitted to an electrosurgical instrument that is operated by a surgeon. The electrosurgical instrument delivers an electrical discharge to cellular matter of the patient's body adjacent to, adjoining with, contiguous with, or juxtaposed to the electrode. The discharge causes the cellular matter to heat up in order to cut tissue and/or cauterize.

The high temperatures involved in electrosurgery can cause charred matter to form and become affixed to the electrode or tip of the electrosurgical instrument. The buildup of charred matter can reduce the efficiency of the cutting and/or cauterizing processes by creating an insulating barrier that interferes with the transference of RF energy to the targeted area. By way of example, when cauterizing an area to prevent bleeding, the charred matter can inhibit the cauterization, cause the destruction of additional tissue, and increase thermal tissue damage. Thus, buildup of the charred matter can slow the surgical procedure, as the surgeon is required to remove the charred matter from the electrode or tip of the electrosurgical instrument.

While the anti-adhesion properties of fluoropolymers that have been used to coat the electrode or tip of an electrosurgical instrument have facilitated electrosurgical cutting and/or cauterizing by reducing the buildup of debris on the electrode or tip, it has not completely eliminated such buildup. Accordingly, it would be an improvement in the art to augment or even replace the fluoropolymer coating with other anti-adhesion materials. Unfortunately, it has heretofore been difficult to adhere other materials to surfaces coated with a fluoropolymer because of anti-adhesion properties.

### BRIEF SUMMARY OF THE INVENTION

The present invention relates to the utilization of a multi-character material in a surface coating. More specifically, the present invention relates to using a multi-character coating material to coat at least a portion of a surface of an electrosurgical instrument in order to provide desirable properties, characteristics and/or attributes at the surface.

Implementation of the present invention takes place in association with a surface, such as at least a portion of the surface of an electrosurgical tip that may be used to cut tissue and/or cauterize blood vessels and/or tissue of a patient during a surgical operation. At least a portion of the tip is coated with a base material, such as a fluoropolymer, to reduce the accumulation of charred blood and/or tissue, known as eschar, at the tip, and to increase the efficiency of the tip to cut and/or cauterize. The tip is also coated with a multi-character material, such as a multi-character material having a hydrophobic character and a hydrophilic character, or a material having a hydrophobic or hydrophilic character and a charged or ionic group, that bonds to the fluoropolymer coating and/or to the tip to provide additional properties, characteristics and/or attributes to the tip.

The multi-character material used to coat a surface of the tip is obtained, for example, through a copolymerization of a hydrophobic monomer or material and a hydrophilic monomer or material such that the resulting copolymer has a hydrophobic characteristic, section or block of material that is separate and distinct from a hydrophilic characteristic, section or block of material. The multi-character material may also include polymers that have a hydrophobic or hydrophilic characteristic, section or block and a unit that is or can be charged. As such, the hydrophobic characteristic or charged unit of the multi-character material is used to bond to the fluoropolymer or base material coating layer, and the hydrophilic region provides desired properties, attributes and/or characteristics to the electrosurgical tip. Optionally, an additional covering layer of a hydrophilic material may be applied over the multi-character material coating layer to increase the desired properties, attributes and/or characteristics at the surface.

In at least one implementation of the present invention, the hydrophilic material used to obtain the multi-character material and/or to provide the optional covering layer is a water-soluble polymer. For example, when a surface coating of an electrosurgical tip includes a water-soluble polymer, water may be attracted to the tip that assists in cooling and/or lubricating the tip. A water-soluble polymer coating layer may further create a low shear, sacrificial layer on the tip that protects and enhances the performance of the tip. Alternatively or additionally, the water-soluble polymer may supply a radical scavenger or inhibitor to reduce damage to thetip, deposit factors or active agents, such as healing factors, from the tip onto one or more contact surfaces of the patient's body, and/or provide other desired properties, attributes and/or characteristics to the tip.

While the methods and processes of the present invention have proven to be particularly useful in the area of electrosurgery, those skilled in the art can appreciate that the methods and processes of the present invention can be used on a variety of different kinds of surfaces and in a variety of different areas of manufacture to yield a coated surface that has desired properties for performing a particular task.

Additional features and advantages of the present invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order that the manner in which the above-recited and other advantages and features of the invention are obtained, a more particular description of the invention briefly described above will be rendered by reference to specific embodiments thereof, which are illustrated, in the appended drawings. Understanding that these drawings depict only typical embodiments of the invention and are not therefore to be considered limiting of its scope, the invention will be described and explained with additional specificity and detail through the use of the accompanying drawings in which:

FIG. 1 illustrates an exemplary system that provides a suitable operating environment for use of the present invention;

FIG. 2A illustrates an exemplary electrosurgical tip for use in the suitable operating environment of FIG. 1 to cut and/or cauterize in general surgery;

FIG. 2B illustrates an exemplary electrosurgical tip for use in the suitable operating environment of FIG. 1 to cut and/or cauterize in particularly dense areas;

FIG. 2C illustrates an exemplary electrosurgical tip for use in the suitable operating environment of FIG. 1 to remove large sections of tissue;

FIG. 2D illustrates an exemplary electrosurgical tip for use in the suitable operating environment of FIG. 1 to cauterize and to seal open structures;

FIG. 2E illustrates an exemplary electrosurgical tip for use in the suitable operating environment of FIG. 1 to isolate tissue and to independently cut or cauterize;

FIG. 3A illustrates an exemplary cross-sectional view of an electrosurgical tip that has a continuous base material coating layer and a multi-character material coating layer applied thereon;

FIG. 3B illustrates an exemplary cross-sectional view of the tip of FIG. 3A that further includes a hydrophilic covering layer to increase the desired properties, attributes and/or characteristics at the tip;

FIG. 4A illustrates an exemplary cross-sectional view of an electrosurgical tip that has a coating layer applied thereon, wherein the coating layer comprises a continuous base material having one or more particles mixed therein;

FIG. 4B illustrates an exemplary cross-sectional view of the tip of FIG. 4A after the tip has been exposed to a sintering process to burn off, thermally degrade and/or evaporate the one or more particles from the base material, leaving pores, channels, tunnels, and/or interconnected pathways in the base material;

FIG. 4C illustrates an exemplary cross-sectional view of the tip of FIG. 4B, where the pores are filled, coated and/or lined with a multi-character material to provide desired properties to the coating layer; and

FIG. 4D illustrates an exemplary cross-sectional view of the electrosurgical tip of FIG. 4C that further includes a covering layer to increase the desired properties, attributes and/or characteristics at the tip.

### DETAILED DESCRIPTION OF THE INVENTION

The present invention relates to the utilization of a multi-character material in a surface coating. More specifically, the present invention relates to using a multi-character material to coat at least a portion of a surface of an electrosurgical instrument in order to provide desirable properties, characteristics and/or attributes at the surface.

In the disclosure, reference is made to using a multi-character material in a surface coating of an electrosurgical instrument. In the disclosure and in the claims the term “multi-character material” shall refer to any material having a hydrophobic characteristic and a hydrophilic characteristic, or a hydrophobic or hydrophilic characteristic and a charged or ionic group. An example of a multi-character material is an amphophilic material.

In the disclosure, reference is also made to the use of a fluoropolymer as a base material coating layer. In the disclosure and in the claims, the term “fluoropolymer” shall refer to a material that includes polymers that contain fluorocarbons or fluorinated hydrocarbons. By way of example, the term “Teflon(t” represents a family of various fluoropolymers.

Reference is also made in the disclosure to particles of a “filler” that may be used to create pores in a base material coating layer. In the disclosure and in the claims the term “filler” shall refer to any polymer material that degrades thermally as the base material is cured, including non-aromatic hydrocarbon polymers. Examples of such filler polymers include polyethylene, polybutylene, polystyrene, polypropylene, and other thermally degrading materials. Additionally, the term “filler” refers to any solid or liquid organic material or solid salt that can be used to create pores in a base material coating layer. Illustratively, an example of an organic filler is an organic solid, liquid or gas that does not dissolve in the base material and which can be evaporated, burned off, or dissolved, such as, but not limited to sugar, wax, oils, fats, organic hydrocarbon liquids, freons, propane, butane, and other like materials. Similarly, an example of an inorganic filler is an inorganic solid, liquid or gas that does not dissolve in the base material and which can be evaporated, burned off, or dissolved, such as but not limited to, salt, silicone oils, carbon dioxide, air, argon and other like materials.

In the disclosure and in the claims the term “pore” shall refer to a cavity that is created or otherwise present in a base material. Moreover, the term “pore” shall include, but is not limited to, a channel, tunnel, interconnected pathway or other cavity in a base material. As will be further explained herein, a pore may be at least partially filled with a multi-character material to provide desired properties, characteristics and/or attributes at a surface of the tip of an electrosurgical instrument, such as attracting water to the surface to assist in cooling and/or lubricating the surface of the tip of the instrument, creating a low shear, sacrificial layer at the surface that protects and enhances the surface, supplying a radical scavenger or inhibitor to the surface to reduce damage at the surface due to gamma sterilization, depositing factors or active agents from the surface onto one or more contact surfaces, and the like. Examples of factors include antibiotic factors, healing factors, antiadhesion factors, anti-tumor factors, tumor necrosis factors, clotting factors, and other medical factors that are beneficial to a patient and are specifically included in the coating of an electrosurgical tip as desired, and by design, for deposition in and on tissues of a patient's body where electrosurgical procedures are performed.

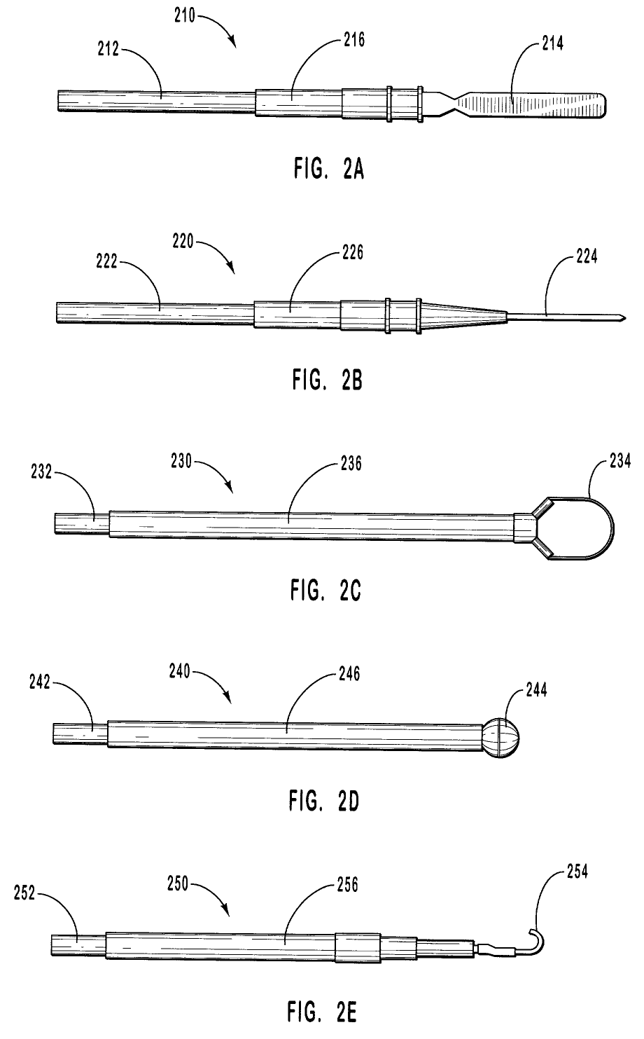

In addition to the above terms, the follow description refers to an “electrode” or “tip,” such terms being interchangeably used herein. The terms “electrode” and “tip” refer to the portion of the electrosurgical instrument that is in contact with the patient and through which the radio frequency energy pass to cut or cauterize the patient's tissue. Examples of “electrodes” or “tips” are depicted in FIGS. 2A-2E, and will be described in greater detail hereinafter.

The following disclosure of the present invention is grouped into three subheadings, namely “Exemplary Operating Environment,” “Applying a Multi-Character Material to a Continuous Base Material Coating Layer of a Surface” and “Applying a Multi-Character Material to a Porous Base Material Coating Layer of a Surface.” The utilization of the subheadings is for convenience of the reader only and is not to be construed as limiting in any sense.

### Exemplary Operating Environment

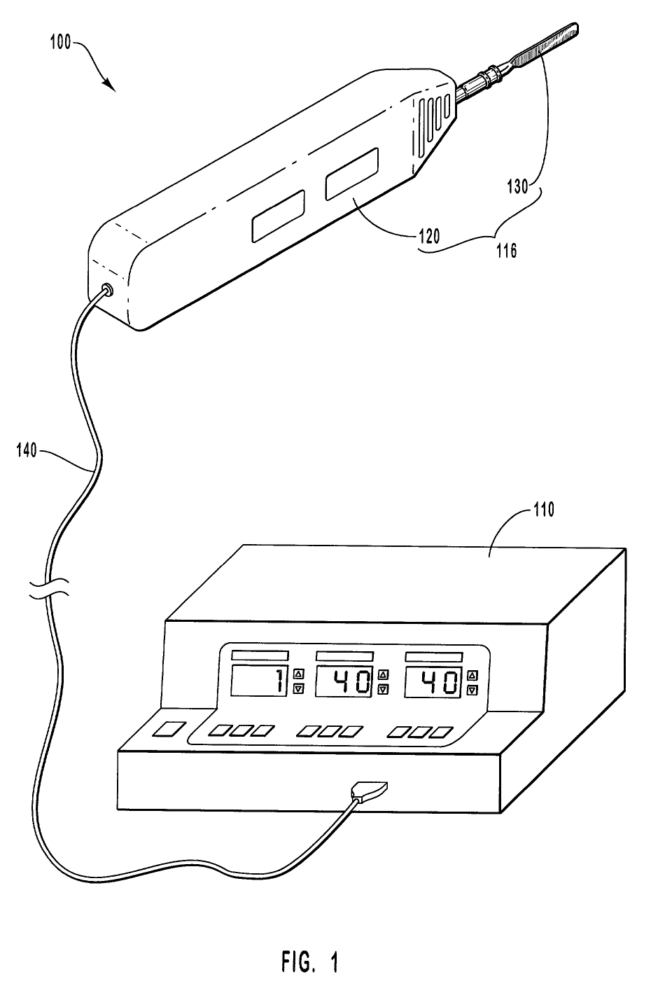

FIGS. 1-2E and the corresponding discussion are intended to provide a brief, general description of a suitable operating environment in which the invention may be implemented. Although not required, the invention will be described in the general context of creating specific properties, attributes and/or characteristics on a surface area of a tip of an electrosurgical instrument. Those skilled in the art, however, will appreciate that embodiments of the present invention may be practiced in association with a variety of different surfaces in order to provide desirable properties, attributes and/or characteristics at the surfaces.

Referring to FIG. 1, an exemplary environment is illustrated that provides a suitable operating environment for use of the present invention. In FIG. 1, electrosurgical instrument system 100 is illustrated, which includes a signal generator 110 and an electrosurgical instrument 116 electrically coupled to signal generator 110 though cord 140. A surgeon typically uses electrosurgical system 100 during surgical procedures to cut tissue and/or to cauterize blood vessels of a patient's body.

In electrosurgery, radio frequency (RF) electrical energy is produced by a signal generator, such as signal generator 110, and is introduced to a patient's body by electrosurgical instrument 116. More specifically, the radio frequency energy is introduced to the patient's body through an electrode or tip 130 electrically coupled to a hand-piece 120; the combination of tip 130 and hand piece 120 forms an exemplary electrosurgical instrument 116. The RF electrical energy generated by signal generator 110 is transmitted from signal generator 110 to tip 130 through hand-piece 120 and cord 140. An electrical discharge is delivered from tip 130 to the patient in order to cause the heating of cellular matter of the patient that is in close contact, adjacent to, juxtaposed, or the like to tip 130. The heating takes place at an appropriately high temperature to allow performance of electrosurgery using hand piece 120 and tip 130. A grounding electrode (not shown) may be employed to carry away any excess charge that dissipated into surrounding tissue of the patient's body.

During electrosurgery, tip 130 may be used to independently or concurrently cut and cauterize the patient's tissue. A constant sinusoidal signal supplied by signal generator 110 and transmitted to electrosurgical instrument 116 to cut through tissue of the patient's body. Alternatively, a damped wave signal supplied by signal generator 110 and transmitted to electrosurgical instrument 116 allows tip 130 to cauterize leaking blood vessels. A combination of the constant sinusoidal signal and the damped wave signal can be supplied by signal generator 110 to tip 130 through hand piece 120 allowing tip 130 to concurrently cut and cauterize, thereby minimizing tissue trauma and blood loss during the surgical procedure.

FIGS. 2A-2E illustrate an exemplary assortment of interchangeable tips or electrodes for use with hand piece 120 to facilitate the acts of cutting tissue and/or cauterizing blood vessels. Each of the interchangeable tips or electrodes has a first end that can be coupled to the hand piece 120, an insulator, and a second end that applies the discharge to the patient's body. The configuration of the second end allows for a great versatility of the acts of cutting and/or cauterizing in a variety of different surgical procedures. Although reference is made to interchangeable tips or electrodes, one skilled in the art can appreciate that alternate embodiments of the present invention can utilizes tips or electrodes that are attached to a hand piece and are non-removable there from.

By way of example, FIG. 2A illustrates tip 210, which is a tip that may be used in general surgery for cutting tissue and for cauterizing blood vessels. End 212 is coupled to the hand piece 120 to allow the RF electrical energy, generated from the signal generator and transmitted to the hand piece 120, to be transmitted through tip 210. A discharge is delivered to the patient's body from end 214, which is in a blade-like configuration. End 214 has two parallel sides that are flat to allow end 214 to function in a similar manner as a traditional scalpel. However, rather than employing a mechanical action for cutting through tissue, the electrical discharge allows end 214 to slide through the tissue as the tissue is being superheated to a high temperature. A coating 216, such as a heat shrink tubing, injection molded polymer, spray applied polymer or paint, or the like combined base material coating layer and a multi-character material, surrounds at least a portion of tip 210 and acts as an insulator.

Similarly, FIG. 2B illustrates tip 220, which may be used for cutting tissue and cauterizing leaking blood vessels in particularly dense or compact areas of a patient's body, such as those experienced in cerebral operations. End 222 is adapted to couple to hand piece 120 to allow the RF electrical energy, generated from the signal generator and transmitted to hand piece 120, to be transmitted to and through tip 220. A discharge is delivered to the patient's body from end 224, which is in a needle-like configuration that comes to a point to allow for very accurate surgical procedures in dense or compact areas of the patient's body. A coating 226 surrounds at least a portion of tip 220 and acts as an insulator. Through the use of tip 220, delicate cerebral tissues can be accurately removed with virtually no damage to any surrounding membranes and with minimal bleeding and/or swelling resulting from the procedure.

FIG. 2C illustrates tip 230, which may be used for the removal of large sections of tissue, as in, for example, prostate and tumor excision. End 232 is adapted to couple to the hand piece 120 to allow RF electrical energy to be transmitted through tip 230. A discharge is delivered to the patient's body from end 234, which is in a loop-like configuration. A coating 236 surrounds at least a portion of tip 230 and acts as an insulator.

FIG. 2D illustrates tip 240, which may be used to specifically cauterize leaking blood vessels and to seal open structures. End 242 is adapted to couple to hand piece 120 to allow RF electrical energy to be transmitted through tip 240. A discharge is delivered to the patient's body from end 244, which is in a spherical configuration. A coating 246 surrounds at least a portion of tip 240 and acts as an insulator.

FIG. 2E illustrates tip 250, which may facilitate a surgeon in reducing extraneous tissue damage by allowing individual tissues or blood vessels to be isolated and independently cut and/or cauterized. End 252 is adapted to couple to hand piece 120 to allow RF electrical energy from the signal generator to be transmitted through tip 250. A discharge is delivered to the patient's body from end 254, which is in a hook-like configuration. A coating 256 surrounds at least a portion of tip 250 and acts as an insulator.

Ends 214, 224, 234, 244 and 254 are examples of surfaces upon which a coating layer may be applied to render one or more desirable attributes and/or properties. When the surface is at least a portion of a tip, an example of a desired property includes minimizing the amount of eschar that accumulates on the tip. The minimization of eschar allows for a more efficient use of the tip by reducing damage to surrounding tissues that may have been otherwise caused by an accumulation of eschar during an electrosurgical procedure.

### Applying a Multi-Character Material to a Continuous Base Material Coating Layer of a Surface

A coating of a surface may include a multi-character material to provide additional properties, characteristics and/or attributes that are desirable to the surface. For example, when the surface is an electrosurgical tip, a base material, such as a fluoropolymer, may be used to provide a coating layer that minimizes the amount of eschar that accumulates on the electrosurgical tip during an electrosurgical procedure, and a multi-character material coating layer may be applied to provide an attraction of water to the tip that assists in cooling and/or lubricating the tip during the procedure. Other examples of properties provided by the multi-character material may include providing a low shear, sacrificial layer on the tip that protects and enhances the performance of the tip, a radical scavenger or inhibitor that reduces damage to the tip due to gamma sterilization, factors or active agents, such as healing factors, that may be deposited from the tip onto one or more contact surfaces of the patient's body, and other such desired properties, attributes and/or characteristics.

The multi-character material may be obtained through a copolymerization of a hydrophobic monomer, polymer, or material and a hydrophilic monomer, polymer, or material such that the resulting copolymer has a hydrophobic characteristic section or block of material that is separate and distinct from a hydrophilic characteristic section or block of material. These sections or blocks of material may be at either the middle or the ends of the resulting block copolymer. A section or block may be as small as one chemical unit, such as a charged hydrophilic group like a sulfate or carboxyl on the end of a less hydrophilic chain. Furthermore, a multi-character material that may be used herein also includes polymers that have a hydrophobic block or a hydrophilic block and a unit on the end that is or can be charged to utilize a technique of electrophoresis, which will be further explained below.

The hydrophobic characteristic or charged unit may be used to bond to the surface of the conductive tip or to the base material coating layer, and the hydrophilic characteristic may provide additional desired properties, attributes and/or characteristics to the surface. Examples of hydrophobic materials include polypropylene oxide (“PPO”), a fluorocarbon, a hydrocarbon, and the like. Examples of hydrophilic materials include water soluble hetero atom polymers (or water soluble heterochain polymers) such as polyethylene oxide (“PEO”), polyethylene glycol (“PEG”), polyethylene oxide-poly (dimethylsiloxane) copolymer (“PEO-PDMS”), other copolymers of ethylene oxide, polylactone, polycaprolactone, other caprolactone copolymers, water soluble nylon, ethylene maleic anhydride copolymer and other maleic anhydrides, ionene (ionic amine) polymers, polyalkylene oxalate; water soluble natural polymers and derivatives thereof such as starch, gelatin, other proteins, chitin-poly(N-acetyl-D-glucosamine) and derivatives, hyaluronic acid and salts thereof, other polysaccharides, chondritic sulfate, agarose, methylcellulose, ethylcellulose, hydroxyethylcellulose, hydroxypropyl-methylcellulose, ethyl-hydroxyethylcellulose, ethyl-methylcellulose, hydroxyethyl-methylcellulose, carboxymethyl cellulose and salts thereof, carboxymethylhy droxyethylcellulose salts, other cellulosic derivatives; water soluble vinyl polymers such as polyvinyl pyrrolidone, polyvinyl alcohol (“PVA”), poly vinyl acetate, ethylene-vinyl acetate copolymers, poly-4-vinyl-n-butylpyridinium bromide, polyvinylmethyl ether, vinylmethyl ether-maleic anhydride copolymer; water soluble acrylic polymers such as poly hydroxyethyl methacrylate, ammonium polyacrylate; water soluble acrylic acid polymers such as poly methacrylic acid; other water-soluble polymers, and the like.

In addition, the multi-character and base materials may be cross-linked together into an interpenetrating network (IPN), or the multi-character material itself may be cross-linked by chemical, photo, or radiation induced methods for enhanced adhesion and durability.

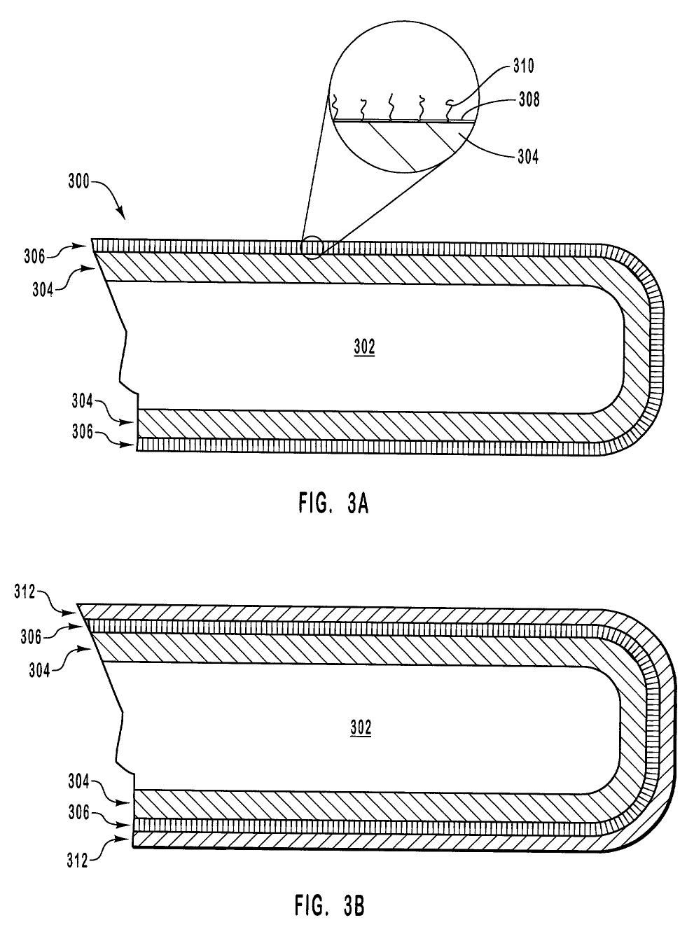

With reference to FIG. 3A, an exemplary cross-sectional view of an electrosurgical tip is illustrated as tip 300, which includes a substrate 302 and a continuous base material coating layer 304. In one embodiment, substrate 302 is a conductive material. For instance, substrate 302 can be a metal, such as surgical stainless steel. A continuous base material coating layer 304 is applied to the substrate 302 of the tip 300. For example, polytetrafluoroethylene (“PTFE”) or another fluoropolymer may be used as the base material to reduce the amount of eschar that develops when tip 300 is used for performing an electrosurgical procedure. Other base materials include a silicone, a ceramic, an aromatic hydrocarbon, an aromatic fluorocarbon, and other similar materials having high temperature stability. Prior to applying the base material coating layer 304 onto tip 300, the surface of substrate 302 may be prepared by being heat cleaned in order to remove any oils or contaminants and may be roughened to provide a footing for the base material.

Once the surface of substrate 302 is prepared, a suspension or emulsion of base material particles and an etching agent is spray coated onto the substrate 302 so as to uniformly apply a coating layer, illustrated as base material coating layer 304, to at least a portion of substrate 302. In one embodiment, the etching agent is naturally included in a base material paint that may be spray coated onto a surface. The etching agent improves the bond between the base material and the substrate 302. As the base material coating layer 304 provides a non-stick coating, the anti-adhesion properties of the base material have heretofore prevented other materials from adhering to the base material. However, in accordance with the present invention, a multi-character material may be used to bond onto at least a portion of the base material coating layer 304 and provide additional desired properties to the tip 300. The multi-character material coating layer 306 may be applied onto the base material coating layer 304 by a spray, dip, brush, or other application process, including by adsorption from a solution or vapor, by wiping, by plasma polymerization, vapor deposition/polymerization, and the like.

In addition, the multi-character and base materials may be cross-linked together into an interpenetrating network (IPN), or the multi-character material itself may be cross-linked by chemical, photo, or radiation induced methods for enhanced adhesion and durability.

With reference to FIG. 3A, an exemplary cross-sectional view of an electrosurgical tip is illustrated as tip 300, which includes a substrate 302 and a continuous base material coating layer 304. In one embodiment, substrate 302 is a conductive material. For instance, substrate 302 can be a metal, such as surgical stainless steel. A continuous base material coating layer 304 is applied to the substrate 302 of the tip 300. For example, polytetrafluoroethylene (“PTFE”) or another fluoropolymer may be used as the base material to reduce the amount of eschar that develops when tip 300 is used for performing an electrosurgical procedure. Other base materials include a silicone, a ceramic, an aromatic hydrocarbon, an aromatic fluorocarbon, and other similar materials having high temperature stability. Prior to applying the base material coating layer 304 onto tip 300, the surface of substrate 302 may be prepared by being heat cleaned in order to remove any oils or contaminants and may be roughened to provide a footing for the base material.

Once the surface of substrate 302 is prepared, a suspension or emulsion of base material particles and an etching agent is spray coated onto the substrate 302 so as to uniformly apply a coating layer, illustrated as base material coating layer 304, to at least a portion of substrate 302. In one embodiment, the etching agent is naturally included in a base material paint that may be spray coated onto a surface. The etching agent improves the bond between the base material and the substrate 302. As the base material coating layer 304 provides a non-stick coating, the anti-adhesion properties of the base material have heretofore prevented other materials from adhering to the base material. However, in accordance with the present invention, a multi-character material may be used to bond onto at least a portion of the base material coating layer 304 and provide additional desired properties to the tip 300. The multi-character material coating layer 306 may be applied onto the base material coating layer 304 by a spray, dip, brush, or other application process, including by adsorption from a solution or vapor, by wiping, by plasma polymerization, vapor deposition/polymerization, and the like.

The multi-character material includes, for example, a hydrophobic characteristic and a hydrophilic characteristic. FIG. 3A illustrates a multi-character material having a hydrophobic characteristic 308 bonded to the base material coating layer 304 and a hydrophilic characteristic 310 extending out there from. A long hydrophobic characteristic region 308 improves the bonding of the multi-character material coating layer 306 to the base material coating layer 304 and increases the longevity of the multi-character material coating layer 306 on the tip 300. In one embodiment, the hydrophilic material used within the multi-character material is a water-soluble polymer, such as PEO, that may attract water to the surface of an electrosurgical tip to assist in cooling and/or lubricating the tip. In one embodiment, a cross-linking or creation of interpenetrating networks (“IPNs”) of the multi-character material by chemical, photo, or radiation induced methods provides enhanced adhesion and durability.

While FIG. 3A illustrates the configuration of the molecular chains in the multi-character material as an elongate hydrophobic characteristic 308 bonded to an elongate hydrophilic characteristic 310, embodiments of the present invention embrace other configurations, such as an elongate hydrophobic characteristic bonded to a bulbous hydrophilic characteristic. In addition, embodiments of the present invention embrace more than one hydrophobic characteristic and/or more than one hydrophilic characteristic in a molecular chain of the multi-character material. Examples of multi-character molecular chains include a PPO group bonded to two PEO groups, a fluorocarbon group bonded to a PEO group bonded to a hydrocarbon group, and the like.

With reference to FIG. 3B, an additional covering layer of a hydrophilic material, such as a water-soluble polymer, may optionally be applied onto the multi-character material coating layer 306 to increase the desired properties, attributes and/or characteristics that are provided at the surface. And, when a hydrophilic material layer 312 is applied, the multi-character material layer 306 assists in bonding the hydrophilic material layer 312 to the substrate 302 even when an anti-adhesive material or fluoropolymer is located therebetween.

### Applying a Multi-Character Material to a Porous Base Material Coating Layer of a Surface

At times, it is advantageous to create pores in the base material coating layer of a surface in order to improve the bond of a multi-character material coating layer. For example, when the base material coating layer of a surface of an electrode or tip includes a material or fluoropolymer having an anti-adhesion property, and a multi-character material is to be applied to the base material coating layer, it may be advantageous to create pores in the base material to improve the adhesion of the multi-character material to the tip. Pores may be created in the base coating layer by inserting desirably sized small particles of a filler, such as a filler polymer, organic filler, or inorganic filler, into the base material prior to applying the base material onto a surface and removing the filler, such as by burn off, thermal degradation, dissolution and/or evaporation, in order to leave pores in the base material coating layer. As provided above, the illustrative embodiment utilizes a filler polymer that may include any material that degrades thermally as the base material is cured, including non-aromatic hydrocarbon polymers. Examples of such filler polymers include polyethylene, polybutylene, polystyrene, polypropylene, and other thermally degrading materials. In fact, nearly any polymer may be used except for thermally stable polymers, such as silicones highly aromatic hydrocarbons, aromatic fluorocarbons, and other fluorocarbon polymers. Once created, the pores may be at least partially filled with the multi-character material to provide the desired properties at the surface, as will be further explained below.

As mentioned above, another example of a filler is an organic solid, liquid or gas that does not dissolve in the base material and which can be evaporated, burned off, or dissolved. Examples of such organic fillers include sugar, wax, oils, fats, organic hydrocarbon liquids, freons, propane, butane, and other like materials. Similarly, another example of a filler is an inorganic solid, liquid or gas that does not dissolve in the base material and which can be evaporated, burned off, or dissolved. Examples of such inorganic fillers include salt, silicone oils, carbon dioxide, air, argon and other like materials.

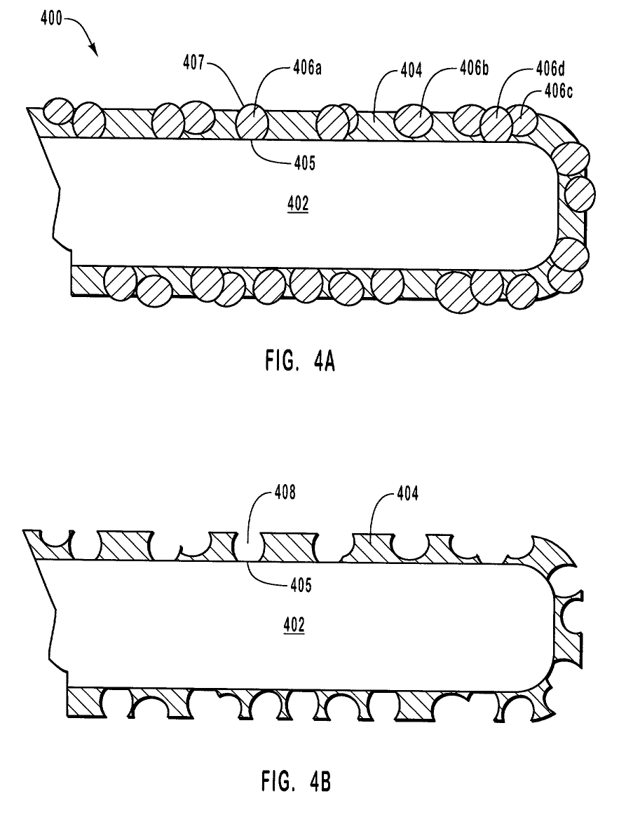

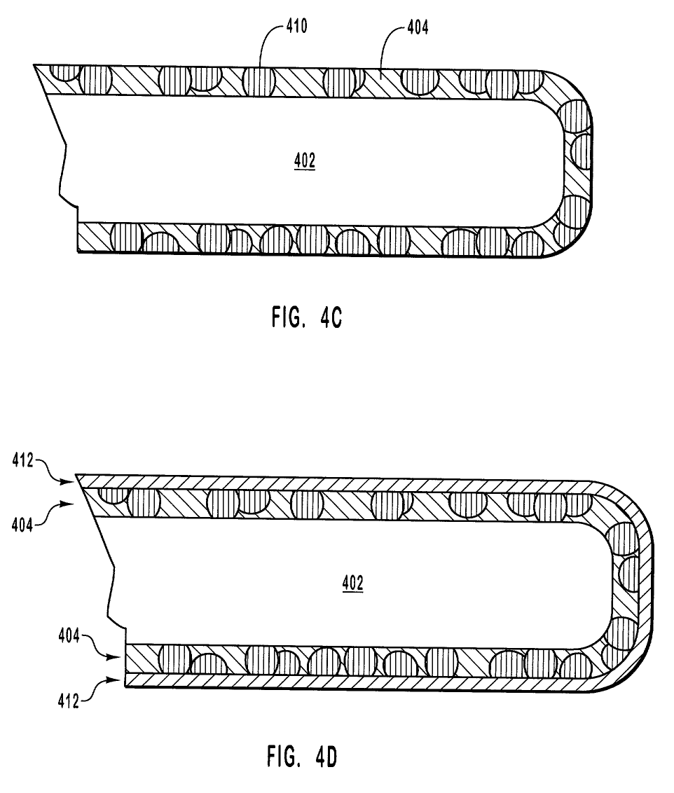

With reference to FIG. 4A, electrosurgical tip 400 is an example of a surface where it may be advantageous to create pores in a base material coating layer to increase the bond of a multi-character material coating layer to tip 400. Once the surface of substrate 402 is prepared, a base material, such as a fluoropolymer, that has previously been mixed with an etching agent and with desirably sized particles of a filler polymer or other suitable filler material is spray-coated onto the tip 400 so as to uniformly apply a coating layer about substrate 402. The etching agent improves the bond between the base material and the substrate 402.

The base material coating layer applied on substrate 402 is illustrated in FIG. 4A as including a plurality of particles 406 of filler polymer, that are distributed within the base material 404. In one embodiment, the pores extend through to the substrate 402 by having the thickness of the coating layer applied onto substrate 402 correspond to the diameter of the particles 406 by the thickness being e.g., equal to or smaller than the diameter of the particles of filler polymer. Therefore, as illustrated in FIG. 4A, a portion of a first end 405 of particle 406a may be in abutting contact with the surface of the substrate 402 and a portion of an opposing end 407 of particle 406a is exposed within the top surface of the base material coating layer. Alternatively or additionally, the particles may not extend through to the surface of the substrate 402. This is illustrated, for example, by particle 406b, which is not in abutting contact with substrate 402. Alternatively or additionally, particles that do not extend through to the substrate may be in contact with particles that do contact the substrate 402. This is illustrated, for example, by particle 406c, which is in abutting contact with a particle 406d that is in abutting contact with substrate 402. Furthermore, while the particles 406 illustrated in FIG. 4A are generally oval in shape and similar in size, the particles of filler, such as a polymer filler, organic filler, or inorganic filler, may have a variety of different shapes and/or sizes, as will be appreciated by those skilled in the art.

Once the base material coating layer has been applied to the substrate 402, a drying process may be employed under controlled humidity to remove, for example, a solvent or etchant material. The tip may then be placed in an oven and heated to quickly evaporate any remaining solvent (e.g. water, MEK, alcohol, etc.) from the tip. The temperature may then be increased to thermally degrade the filler, so as to remove the particles 406, and reach a sintering temperature. Alternatively, if the filler does not thermally degrade, such as when the filler is a salt, it may be dissolved away for example by soaking the tip in water or other suitable solvent.

FIG. 4B illustrates the electrosurgical tip after having been subject to the sintering process. This sintering process evaporates any carrier liquid remaining from the spray up, coating or etching material (which may be the first to evaporate from the tip), causes the base material to enter into any cracks located on the surface of substrate 402, and coalesces any remaining particles of the base material. As provided above, the sintering process further causes the thermal degradation of the filler and causes evaporation of the degradation products so as to create pores in the base material coating layer once occupied by the particles. In FIG. 4B, a plurality of pores 408 are illustrated that extend into and/or through the base material coating layer to cause the base material coating layer to be porous. The size of the pores corresponds to the size of the particles of filler polymer originally selected for inclusion into the base material.

With reference to FIG. 4C, the pores may be at least partially filled, coated or lined with a multi-character material to provide additional desired properties to the coated surface, including the ability of a hydrophilic material to adhere to at least a portion of the filled, coated or lined pore. Since the pores were created so as to extend into and/or through the base material coating layer, the hydrophobic characteristic of the multi-character molecular chain may adhere directly onto the substrate and/or to the base material. Therefore, FIG. 4C illustrates the filled pores as being interspersed within a coating layer of base material 404 on substrate 402. In one embodiment, the pores are at least partially filled with the multi-character material through the process of electrophoresis. Other embodiments embrace the acts of adsorption from solution or vapor, spraying, wiping, or other methods to apply the multi-character material into at least a portion of the pores 408.

Electrophoresis is the movement of electrically charged particles through or along a medium as a result of an electric field formed between electrodes immersed in the medium. In this process, a charge may exist at one or both ends and/or within the molecular chain of a multi-character material. The charged molecular chain is then electrophoresed using an electric field to attract the multi-character material into the pores created in the base material, resulting in at least partially filled pores, such as filled pore 410 of FIG. 4C, that are interspersed in the coating layer of base material 404 to provide additional properties, characteristics and/or attributes to the coated surface.

In another embodiment of the present invention, rather than creating pores in a coating layer, the pores already exist, such as when the substrate of the electrosurgical tip comprises a porous metal (not shown). As such, one or more of the pores of the porous metal are at least partially filled with the multi-character material to provide the desirable properties to the surface of the tip. In this embodiment, the process of electrophoresis or another application method may be employed to apply the multi-character material into the pores of the porous metal, resulting in at least partially filled pores located at the metallic substrate of the tip that are interspersed in the porous metal.

With reference to FIG. 4D, an additional covering layer may optionally be applied through a dip, spray, brushing, or other application process, and is illustrated as covering layer 412. The covering layer may include a hydrophilic material, such as a water-soluble polymer, and/or a multi-character material and may be applied to increase the desired properties at the coated surface. In addition, when a hydrophilic material is applied, the multi-character material assists in bonding the hydrophilic material to the substrate 402 even when an anti-adhesive material, such as a fluoropolymer, is located therebetween.

Thus, as discussed herein, the embodiments of the present invention embrace the application and utilization of a multi-character material on a surface. The hydrophobic characteristic or the charged unit of the multi-character material assists in deposition and/or bonding to the base material and/or conductive substrate and the hydrophilic characteristic may introduce desirable properties, characteristics and/or attributes to the surface. By way of example, when the surface is an electrosurgical tip, the multi-character material and optional hydrophilic coating layer may cause an attraction of water to the surface, even when the surface may otherwise be hydrophobic, such as when the surface includes a fluoropolymer coating. The attraction of water may assist in cooling the tip by evaporation during use of the electrosurgical instrument, thereby protecting and prolonging the service life of the base material on the tip. The attraction of water may also act as a lubricant for an enhanced release character of the base material. The presence of the multi-character material and/or an optional hydrophilic coating layer may provide a low shear, sacrificial layer during tip cleaning that can serve to protect and enhance the activity of the base material, and/or may provide a radical scavenger or inhibitor to reduce damage done to the base material during a process of gamma sterilization, thereby improving the properties and service life of the base material. Furthermore, the multi-character material and/or optional hydrophilic coating layer may act as a carrier of factors or active agents, such as antibiotic factors, healing factors, anti-adhesion factors, anti-tumor factors, tumor necrosis factors, clotting factors, and other medical factors that are beneficial to a patient and are specifically included in the coating of the tip as desired, and by design, for deposition in and on tissues of a patient's body where electrosurgical procedures are performed.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed and desired to be secured by United States Letters Patent is:

1. An electrosurgical instrument, the electrosurgical instrument comprising:
a hand piece configured to receive radio frequency energy from a electrosurgical generator; and
a conductive tip adapted to receive the radio frequency energy from the hand piece, the conductive tip comprising, on at least a portion of the tip, a multi-character material.

2. An electrosurgical instrument as recited in claim 1, wherein the coating further comprises a base material interposed between at least a portion of the multi-character material and a surface of the tip.

3. An electrosurgical instrument as recited in claim 2, wherein the coating further includes an etching agent.

4. An electrosurgical instrument as recited in claim 2, wherein the base material forms a continuous coating layer on at least a portion of the tip.

5. An electrosurgical instrument as recited in claim 4, wherein the base material includes one or more pores therein and the multi-character material occupies at least a portion of the pores.

6. An electrosurgical instrument as recited in claim 1, wherein the multi-character material comprises an amphophilic material with molecular chains having a hydrophilic characteristic, which comprises a water-soluble polymer, and a hydrophobic characteristic.

7. An electrosurgical instrument as recited in claim 2, wherein the base material comprises at least one of:
(i) a fluoropolymer;
(ii) a silicone;
(iii) a ceramic;
(iv) an aromatic hydrocarbon;
(v) an aromatic fluorocarbon; or
(vi) a porous metal.

8. An electrosurgical instrument as recited in claim 7, wherein the water-soluble polymer comprises at least one of:
(i) polyethylene oxide;
(ii) polyethylene glycol; or
(iii) a copolymer of ethylene oxide.

9. An electrosurgical instrument as recited in claim 7, wherein the water-soluble polymer comprises at least one of a water soluble hetero atom polymer, a water soluable acrylate polymer, a water soluble acrylic acid polymer, a water soluable vinyl polymer, and a water soluble natural polymer.

10. An electrosurgical instrument as recited in claim 7, wherein the coating further includes a radical scavenger to reduce damage to the coating during a process of gamma sterilization.

11. An electrosurgical instrument as recited in claim 7, wherein the hydrophobic characteristic comprises at least one of:
(i) polypropylene oxide;
(ii) a fluorocarbon; or
(iii) a hydrocarbon.

12. An electrosurgical instrument as recited in claim 7, wherein the water-soluble polymer is a carrier that provides a factor on a contact area of a patient's body during the electrosurgical procedure.

13. An electrosurgical instrument as recited in claim 12, wherein the factor includes at least one of:
(i) an antibiotic factor;
(ii) a healing factor;
(iii) an anti-adhesion factor;
(iv) an anti-tumor factor;
(v) a tumor necrosis factor; or
(vi) a clotting factor.

14. An electrosurgical instrument as recited in claim 7, wherein the water-soluble polymer provides a low shear, sacrificial layer to the tip.

15. An electrosurgical instrument as recited in claim 1, wherein the tip includes a porous metal.

16. An electrosurgical instrument as recited in claim 1, wherein the multi-character material includes a charged unit.

17. A tip adapted for use in performing an electrosurgical procedure, the tip comprising:
a prepared surface; and
a coating applied to at least a portion of the prepared surface, wherein the coating includes a multi-character material.

18. A tip as recited in claim 17, wherein the prepared surface is a substrate that comprises a porous metal.

19. A tip as recited in claim 17, wherein the prepared surface is a substrate that comprises a roughened metal.

20. A tip as recited in claim 17, wherein the prepared surface is a substrate that comprises surgical stainless steel.

21. A tip as recited in claim 17, wherein the multi-character material includes an amphophilic material with molecular chains having a hydrophilic characteristic and a hydrophobic characteristic, and wherein the hydrophilic characteristic comprises at least one of:
(i) polyethylene oxide;
(ii) polyethylene glycol; or
(iii) a copolymer of ethylene oxide.

22. A tip as recited in claim 17, wherein the coating further comprises a base material.

23. A tip as recited in claim 22, wherein the base material comprises a fluoropolymer.

24. A tip as recited in claim 22, wherein the multi-character material comprises a radical scavenger that reduces damage to the coating during a process of gamma sterilization.

25. A tip as recited in claim 17, wherein the multi-character material provides a low shear, sacrificial layer.

26. A tip as recited in claim 17, wherein the multi-character material comprises a charged unit.

27. A tip as recited in claim 17, wherein the multi-character material comprises a carrier that provides a factor to the contact area of a patient during an electrosurgical procedure.

28. A tip as recited in claim 27, wherein the factor comprises at least one of:
(i) an antibiotic factor;
(ii) a healing factor;
(iii) an anti-adhesion factor;
(iv) an anti-tumor factor;
(v) a tumor necrosis factor; or
(vi) a clotting factor.

29. A method for coating a tip of an electrosurgical instrument, the method comprising the acts of:
preparing a surface of an electrosurgical tip to be coated; and
applying a multi-character material coating layer on the surface.

30. A method as recited in claim 29, wherein the surface is first coated with a base material coating layer.

31. A method as recited in claim 30, wherein the base material coating layer comprises one or more pores, and wherein the multi-character material coating layer occupies at least a portion of the pores.

32. A method as recited in claim 31, wherein the act of applying a multi-character material comprises the act of using a process of electrophoresis to draw the multi-character material into at least a portion of the pores, wherein the combination of the multi-character material and the base material form a first layer about at least a portion of the tip.

33. A method as recited in claim 32, wherein the multi-character material comprises a charged unit.

34. A method as recited in claim 32, wherein the base material comprises a fluoropolymer.

35. A method as recited in claim 34, further comprising the act of applying a coating layer onto the first layer, wherein the coating layer includes a hydrophilic material.

36. A method as recited in claim 29, wherein the act of applying a multi-character material includes utilizing an application process that comprises at least one of:
(i) a dip process;
(ii) a spray process
(iii) a brushing process;
(iv) a wiping process; or
(v) an adsorption process.

37. A method as recited in claim 36, wherein the multi-character material comprises a multi-character with molecular chains having a hydrophobic characteristic and a hydrophilic characteristic, and wherein the hydrophilic characteristic comprises at least one of
(i) polyethylene oxide;
(ii) polyethylene glycol; or
(iii) a copolymer of ethylene oxide.

38. A method as recited in claim 37, wherein the hydrophobic characteristic comprises at least one of:
(i) polypropylene oxide;
(ii) a fluorocarbon; or
(iii) a hydrocarbon
